(19) United States
(12) Patent Application Publication
(10) Pub. No.: US 2009/0105710 A1
(43) Pub. Date: Apr. 23, 2009

(54) Mechanical Distractor To Facilitate Surgery And Evaluation Of Joints

(76) Inventors: Charles Saltzman, Salt Lake City, UT (US); Annunziato Amendola, Coralville, IA (US)

Correspondence Address: FOLEY & LARDNER LLP, 150 EAST GILMAN STREET, P.O. BOX 1497, MADISON, WI, 53701-1497, US

(21) Appl. No.: 12/256,263

(22) Filed: Oct. 22, 2008

Related U.S. Application Data
(60) Provisional application No. 60/981,879, filed on Oct. 23, 2007.

(51) Int. Cl. A61B 17/58; A61F 5/00; A61B 17/00

(52) U.S. Cl. 606/90; 602/32; 606/59

(57) ABSTRACT

A medical apparatus having a tensioning device secured to a fixed point; a wire or pin inserted transversely through a bone; and a tension line connecting the tensioning device to the wire or pin. The tension line transmits a force from the tensioning device to the wire or pin to facilitate distraction of a joint of a subject.

### CROSS-REFERENCE TO RELATED APPLICATIONS

This application claims the benefit of U.S. Provisional Application No. 60/981,879 filed Oct. 23, 2007, the entire contents of which are incorporated herein by reference.

### FIELD

The present invention generally relates to the field of medical apparatuses. More specifically, the invention is directed to an apparatus for distraction of a joint.

### BACKGROUND

Many disorders of the joints can be treated by limited surgical approaches and arthroscopic means. Yet, it is difficult to perform such procedures in a way that permits visualization of the problem region without damaging normal structures that are both in and surrounding the joint. For example, the devices for use in facilitating visualization of the ankle and subtalar joint can damage the musculature and tendons of the foot and ankle, or the soft tissues in the areas above and below the ankle joint. Additionally, the devices used to distract the hip can damage the skin and musculature of the leg, and the devices used to distract the should can damage the skin and musculature of the arm.

With respect to distraction of the ankle and subtalar joint, the distraction devices can be divided into two main classes: those that wrap cloth around the foot and apply a force, or those that require pins to be placed above and below the joints to distract the joint. The cloth wrapping devices have at least three major drawbacks. First, the wrapping flexes the ankle so the foot is positioned downward which tightens the ankle joint capsule. Second the wraps are often in the way of the instruments used to treat the ankle or subtalar joint. Third, the force applied must be limited as it can cause significant skin or nerve problems. The pin placement devices require that one or more pins be placed above and below the ankle and/or subtalar joint. The pins are then connected to an external device that forces the pins apart to distract the joint. There are several reasons that limit the utility of this approach. First, placing pins through the soft tissues above the ankle may cause damage to the bones, muscles, tendons and nerves. Second, such distractor apparatuses are often in the way of the instruments used to perform the surgery. Third, the ankle or foot is not fully immobilized in space to give 360° access to the foot and ankle.

While the cloth wrapping and pin devices have enjoyed some success, the use of each is limited as they do not allow unfettered and full access to the joints of the ankle and foot. For example, such methods require a surgical assistant to hold the foot solidly or move it into a position that is conducive to surgical intervention, however the surgical assistant may then become yet another obstacle for the surgeon that is treating or examining the distracted joint.

In addition to the ankle and subtalar joint, other joint arthroscopies are hindered by the presently used devices. For example, hip distraction is typically performed by wrapping the ankle and lower leg in cloths and tapes which are then connected to a distraction apparatus. However, as with the cloth wrapping devices for ankles, this can put undue strain on the skin and musculature of the ankle and lower leg. Similar wrappings are also used for elbow and shoulder distraction.

Therefore, there exists a need for a medical apparatus to distract joints, which minimize skin and tissue damage, allows for full visualization, in-line distraction, gradual force application, immobilization of the extremity, and which allows for 360° access to the joint that is distracted.

### SUMMARY

In one aspect, a medical apparatus is provided including a tensioning device, a wire or pin inserted transversely through a bone, and a tension line connecting the tensioning device to the wire or pin, where the tension line transmits a force from the tensioning device to the wire or pin to facilitate distraction of a joint of a subject. In some embodiments, the tensioning device is secured to a fixed point. In some embodiments of the medical apparatus, the fixed point is a surgical table upon which the subject is positioned, an attachment to the surgical table upon which the subject is positioned, a stand, a floor, a ceiling, or a wall. In other embodiments of the medical apparatus, the fixed point is a stand and the stand is connected to a surgical table, a floor, a ceiling, or a wall.

A large variety of joints may be distracted by the medical apparatus. In some embodiments, the joint is an ankle, a subtalar joint, a talonavicular joint, midfoot joint, or a forefoot joint. In some embodiments, the bone is a bone of the hindfoot. In some such embodiments, the bone of the hindfoot is a talus or a calcaneus.

In other embodiments, the tensioning device includes a pulley, a manual winch, an electronic winch, a pneumatic piston, a pneumatic winch, a hydraulic piston, a hydraulic winch, a block-and-tackle, a weight system, or a combination of any two or more thereof. In some embodiments, the tensioning device includes a locking mechanism. In some such embodiments, the locking mechanism is a ratchet or a clamp.

In some embodiments, the medical apparatus further includes a clamp to connect the wire or pin to the tension line. In some such embodiments, the clamp is a yoke, a traction bow, or a traction bale.

In some embodiments, the joint is a joint of the ankle or foot. In other embodiments, the joint is a hip. In other embodiments, the joint is ajoint of the knee, shoulder, elbow, foot, toe, hand, finger, or wrist. In other embodiments, the bone is a bone of the ankle or foot. In other embodiments, the bone of the foot is a talus or a calcaneus. In other embodiments, the bone is a bone of the leg. In other embodiments, the bone of the leg is a femur.

In another aspect, a method of distracting a joint using a medical apparatus embodied herein is provided, including: inserting the wire or pin transversely through the bone; connecting the pin to the tensioning device via the tension line; and tensioning the tension line to distract the joint of the subject. In some embodiments the method further includes securing the tensioning device to a fixed point. In some embodiments, the fixed point is a stand. In some embodiments, the method further includes adjusting the stand to a predetermined height and a predetermined distance from a surgical table. In other embodiments, the method further includes securing the subject to a surgical table. In other embodiments, the method further includes connecting the wire or pin to a clamp and connected the clamp to the tension line.

In another aspect, a surgical drape is providing including a cut-out surgical field, a first face and a second face, the second face having an adhesive region proximal to the cut-out surgical field. In some embodiments, the drape is configured to be gathered and tied under a surgical site such that the second face forms a basin. In other embodiments, the surgical drape also includes a fluid transfer line.

### BRIEF DESCRIPTION OF THE DRAWINGS

FIG. 1 is an illustration of a distractor according to one embodiment.

FIG. 2 is an alternative illustration of the distractor shown in FIG. 1.

FIG. 3 is an alternative illustration of the distractor shown in FIG. 1.

FIG. 4 is an illustration of a yoke attached to a tension line and to a wire or pin, where the wire or pin is inserted through a bone in the hindfoot of a subject.

FIG. 5 is an illustration of a distractor showing a yoke attached to a tension line and a wire or pin, where the wire or pin has been inserted through a bone in the hindfoot of a subject, according to one embodiment.

FIG. 6A is an illustration of a traction bow, wire/pin, secondary connector, and hook, according to one embodiment.

FIG. 6B is an detailed view of the ends of the traction bow where the wire/pin is connected, according to one embodiment.

FIG. 7 is a side view illustration of a weight and pulley system, according to one embodiment.

FIG. 8 is an illustration of table mounted tensioning device, according to one embodiment.

FIG. 9 is a top view of a surgical drape, according to one embodiment.

FIG. 10 is a side view of a surgical drape defining a surgical field and gathered to form a basin, according to one embodiment.

### DETAILED DESCRIPTION

In one aspect, a medical apparatus is provided for the distraction of a joint of a subject. In some embodiments, the joint is an ankle, although the use of the medical apparatus for the distraction of the subtalar joint, or other joints of the body will be readily envisioned by those of ordinary skill in the art. For example, the medical apparatus may be used to distract the joints of the ankle, knee, hand, foot, hip, shoulder, elbow, finger, wrist, or other joints. The medical apparatus allows for a surgeon, or other user of the medical apparatus, to evaluate and treat problems with joints. The medical apparatuses embodied herein apply a force to a bone through a tensioned wire or pin. The force is supplied to the tensioned wire or pin by a tensioning device. The tensioning device may be secured to a fixed feature in a room. The force supplied is sufficient to cause the distraction of a joint. Where the joint to be distracted is an ankle, such medical apparatuses can securely suspend the foot or ankle in space, thus allowing the surgeon 360° access to the joint.

Tensioning devices are those devices that are capable of applying, or assisting in the application of, a force to a tension line. For example, the tensioning device may be a weight and pulley system, a winching system, a block-and-tackle, a combination of any two or more thereof, or other device known to those of skill in the art capable of applying a force. In some embodiments, an amount of force to be applied by the tensioning device is an adjustable amount. In other embodiments, the force may be readily released.

As described herein the tension line may be any of a number of such materials known to those of skill in the art. For example, tension lines include straps, cables, ropes, and the like. The materials may be synthetic materials such as nylon, Kevlar, and other polymers and polymer blends; metals or cables of steel and/or stainless steel; and/or natural materials; or blends and/or mixtures of any two or more thereof.

A surgeon, or other operator of the medical apparatus, needs to apply as much force to a joint as is needed. In some cases, the amount of force applied is sufficient for the distraction of the joint. The actual amount of force needed may be different depending upon the joint to be distracted, the patient, the amount of distraction of the joint required, or other factors known to those of skill in the art. In some embodiments, the force is from about 5 to about 250 pounds, from about 10 to about 225 pounds, from about 20 to about 200 pounds, from about 30 to about 200 pounds, from about 40 to about 175 pounds, from about 50 to 150 pounds, or from about 50 to about 125 pounds. In some embodiments, the tensioning device includes an overload protection device that can limit the amount of force that is applied to a given joint. As used herein, an overload protection device refers to a device that limits the amount of force that may be applied to a joint by the tensioning device to which it is attached. For example, if a maximum amount of tension is to be 100 pounds, the overload protection device will not allow the tensioning device to apply more then 100 pounds. In some embodiments, the overload protection device is variable such that the maximum force that may be applied by the tensioning device can be adjusted over a range of values.

In some embodiments, the tensioning devices also include a locking mechanism. In some embodiments, the locking mechanism holds the applied force at a constant amount, to be determined by the surgeon or other operator of the medical apparatus. The locking mechanism is designed to aid in controlling the force applied to the tension line and prevent unintended release of the force. Such locking mechanisms may include a ratchet, a clamp, or other locking mechanism known to those of skill in the art.

The tensioning device is typically secured to a fixed point. In some embodiments, the fixed point is a surgical table upon which the subject undergoing joint distraction is positioned, or it may be a point that is associated with a fixed feature of the room in which the distraction is being conducted such as the surgical table, floor, ceiling, wall, and the like.

In some embodiments, the fixed point is a stand or other device that is connected to the surgical table, or is connected to a fixed feature of the room. The stand may provide a fulcrum for the force from the tensioning device. In such embodiments, the stand may be adjustably connected to the surgical table or fixed feature of the room. For instance, the stand may be connected to the surgical table, such that the stand may be removed from the surgical table, or the stand and the surgical table may be connected via a rigid connector and connector beam such that the distance of the stand and tensioning device from the table may be adjusted and secured in place. Other devices that may be used as the fixed point, include, but are not limited to, devices such as an orthopedic positioning unit. Such units are known in the art, one example being the Spider unit available from Tenet Medical Engineering of Alberta, Canada.

The tensioning device may be connected to the stand, or other device, via a plate or other attachment device known to those of skill in the art. Suitable stands for use in the disclosed medical apparatuses typically have a vertical member to which the tensioning device is attached and a base. As described, the base is a support point for the vertical member and also may serve as a point of attachment of the stand to the surgical table through the connection beam or beams. In some embodiments, the base of the stand has a number of legs that extend toward the surgical table, thus providing stability to stand when the tension line is tensioned from the tensioning device to the subject. Such legs extending toward the table aid in preventing the stand from tipping toward the subject when the tension line is tensioned. The base may be round, square, or have 1, 2, 3, 4, 5, or more legs.

Occasionally, the force imparted by the tensioning device is sufficient to cause the stand to be lifted from the ground and tip toward the subject undergoing distraction. To minimize or prevent such action from occurring, in some embodiments, weights are attached to, or laid across, the stand or base of the stand to provide additional stability to the stand.

In some embodiments, the tensioning device may be a weight and pulley system. In such a system, at least one pulley is secured to the fixed point and the tension line extends from the wire or pin and to the at least one pulley, such that when weights are attached to the tension line a force is transmitted along the tension line, to the pulley, and to the wire or pin. One or more additional pulleys may be added to re-direct the tension line for the force applied. The amount of weight may then be varied to vary the tension required for distraction of the joint. For example, the weight may be applied as a single weight of an appropriate mass, or a series of weights may be added to vary the mass and thereby vary the force applied to the tension line. The weight or weights may be attached directly to the tension line, or a weight attachment device may be attached to the tension line and the weight or weights may then be attached to the weight attachment device.

In some embodiments, the tensioning device may be a winching system. In such a system, a winch is secured to the fixed point and the tension line extends from the wire or pin to a spindle of the winch. In some embodiments, a handle operates the winch to tension the tension line and apply a force to the wire or pin. In other embodiments, the winch is driven by an electronic motor. In yet other embodiments, the winch is driven by an electronic motor that can be activated by a foot switch so that operation of the tensioning device is at least partially hands-free. Suitable winches are those that are typical of a winch such as a boat trailer winch, or a vehicle winch, and also winches commonly known as a “come along.”

In some embodiments, the tensioning device may be a block-and-tackle system. In such a tensioning device, a series of pulleys are used to apply, or assist in the application of, a force to a tension line connected to the wire or pin. The pulleys in such block-and-tackle systems may be compound pulleys with more than one pulley on a common spindle. Devices such as a block-and-tackle may decrease the applied force needed to achieve sufficient force to distract a joint, by using the cumulative properties of multiple pulleys. In some embodiments, a winch may be used to apply a force to the tension line which extends from the winch, through the block and tackle system, and to the wire or pin. In other embodiments, the tension line is tensioned by hand to apply a force to the tension line, through the block-and-tackle and to the wire or pin. The tension is then adjusted by the block and tackle. In such embodiments, the tension line may then be secured by a clamp, holes in the tension line that may be positioned on a peg, or other securing device known to those of skill in the art.

In some embodiments, the tensioning device is pneumatically or hydraulically driven. In such embodiments, the tensioning device may be a pneumatic (air-driven) or hydraulic (fluid driven) device that, when activated, provides an applied force to a tension line le that transmits that force to the joint that is to be distracted. For example, a pneumatic or hydraulic tensioning device may be a piston, a winch, or other such device known to those of skill in the art. As with the other tensioning devices embodied herein, pneumatic or hydraulic tensioning devices may also include an overload regulator to reduce the chance that too much force is applied to a particular joint.

In other embodiments, the height of the stand is adjustable to accommodate different surgical environments such as different table heights or the preferences of different surgeons. The stand may provide for adjustment of a vector of the force applied from the tensioning device. Typically the force is applied in a plane that is either the same as, or parallel to, a plane of the surgical table top. However, certain applications may require that the force be applied in a different plane and the height adjustment of the stand may be used to accommodate such other vectors. In embodiments, where the tensioning device is connected to the stand via a plate, the plate may be connected to the stand with a clamping device that allows for positional adjustment of the plate on the stand.

The wire or pin of the medical apparatus is inserted through a bone. The wire or pin is typically inserted through a bone having good structural stability and is capable of holding up against the force that is applied by the tensioning device without deteriorating or breaking. In embodiments where the medical apparatus is for distraction of an ankle or subtalar joint, the bone is a bone of the hindfoot, such as the calcaneus. The wire or pin is placed through a bone or bones that is located between the joint and the tension line, such that a force that is applied to the wire or pin will result in distraction of the joint. The wire or pin is typically inserted transversely through the bone rather than longitudinally into the bone, although in some applications such longitudinal insertion may be done. In such cases, the wire or pin can be threaded and inserted into the bone in a screw-like fashion.

The wire or pin may be made from a metal or metal alloy, such as but not limited to stainless steel, titanium and titanium alloys, chromium and chromium alloys, nickel and nickel alloys, or molybdenum and molybdenum alloys. The wire or pin, generally has a diameter of about 1 mm to about 2 mm. The surface of the wire or pin can be smooth, threaded, or shaped to be engaged by the clamp, yoke, or other connector. The wire or pin is placed through the bone for the purposes of joint distraction, and then removed at the end of the arthroscopic procedure. Depending upon the joint to be distracted, the wire or pin may be of varying lengths or of other diameters to accommodate more or less force that needs to be applied. Wire/pin dimensions suitable for distraction of the intended joint will be readily determined by those of skill in the art.

In some embodiments, the clamp, yoke, or other connector is used to secure the tension line to the wire or pin. Such a clamp, yoke, or other connector known to those of skill in the art, provides a connection of the tension line to the wire or pin after insertion into the bone. For example, suitable clamps and/or yokes include, but are not limited to, foot frames used for thin wire external fixation, traction bows, and traction bales, as are known to those of skill in the art.

In one embodiment, the clamp may be a generally, Y-shaped yoke where the yoke has a proximal end and a distal end, the distal end having two branches, a first branch and a second branch, spaced apart from one another. The proximal end may then be attached to the tension line, while the first branch is secured to a first end of the wire or pin, and the second branch is secured to the second end of the wire or pin. Thus, when the wire or pin is inserted through a bone in the foot, the branched portion of the yoke acts as a harness applying a force equally to the first and second ends of the wire or pin, when the force is transmitted to the yoke through the tension line, from a tensioning device, and through the proximal end of the yoke.

In another embodiment, attachment of the tension line to the wire or pin may also be accomplished using a generally U-shaped yoke. A first and second end of the open end of the U-shaped yoke are respectively attached to a first end and a second end of the wire or pin, and the bottom of the U-shaped yoke is a point of attachment of the yoke to the tension line. Shapes other than Y- or U-shaped yokes or clamps may be readily envisioned by those of skill in the art.

The clamp/yoke or other connector typically has a robust connection to the wire or pin that will withstand the tensioning of the tension line. In some embodiments, the first branch or open end of the clamp/yoke has a bolt that is inserted transversely through it and is secured by a nut, and optionally a washer. The wire or pin is then secured between a head of the bolt and the clamp/yoke, the washer and the clamp/yoke, or the nut and the camp/yoke. After tensioning of the wire or pin across the clamp/yoke and through the bone, the second end may be secured in a similar fashion. In another embodiment, the first and second branches or open ends of the clamp/yoke are slotted to receive the wire or pin. In such embodiments, the ends of the wire or pin are inserted into the slots and screws are then turned across the open slot to clamp onto the wire or pin.

As with many surgical instruments, the clamp, yoke, or other connector is typically sterilized prior to use, whereas the tensioning device and/or the tension line may or may not be sterilized. To preserve the sterility of the surgical field in which the clamp, yoke, or other connector is used, an optional secondary connector may be inserted between the clamp, yoke, or other connector and the tension line. The optional secondary connector is also typically sterilized to provide a sterile transition from the surgical field to the non-sterile area outside of the surgical field. In some embodiments, the optional secondary connector is a connector that is configured to engage both the tension line and the clamp, yoke, or other connector. Such secondary connectors may provide additional options to the surgical team by providing a pivoting point for twists in the line to be relieved without putting pressure on the yoke and pin attached to the subject, or may provide a more facile manner of connecting the tension line to the yoke via an S-hook, C-clamp, or other connection that may be easily engaged and disengaged.

In another aspect, a surgical drape is provided. The surgical drape has a cut-out surgical field to allow access to the joint that is to be distracted and the distraction apparatus. The surgical drape also allows for access to the tensioning device, and the drape is configured to collect fluids, thereby keeping the floor dry and preventing the fluids from contaminating the surrounding area.

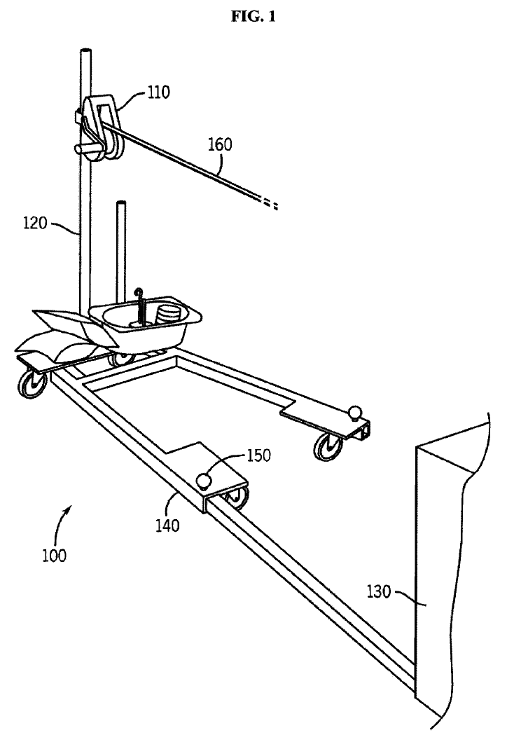

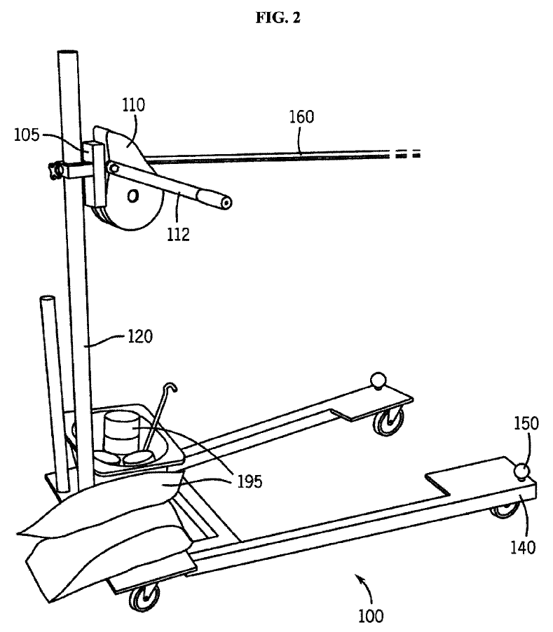

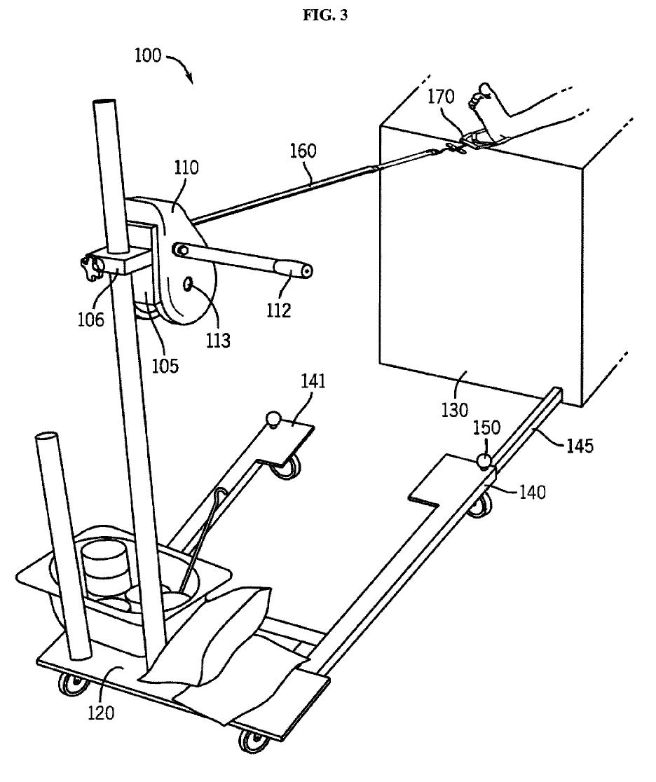

Referring now to FIGS. 1-3, a medical apparatus 100 is shown. In medical apparatus 100, a tensioning device is exemplified as a winch 110. The winch 110 has a handle 112 and a spindle 113, and is supported by a stand 120 adjustably connected to a surgical table 130 via connector 140 and a connector beam 145. Once the adjustable connection of the stand 120 to the surgical table 130 is set, the position may be maintained via a set-screw 150, a bolt, a bayonet-style connection, a spring-loaded pin and hole connection, or other position maintaining mechanism. A tension line 160 is connected to winch 110. The winch 110 is connected to the stand 120 via a plate 105 and a clamp 106. Additional connectors 141 may be provided to either adjust for the preference of the surgeon as to the side of attachment of the stand 120 to the surgical table 140, or for additional securement of the stand 120 to the surgical table 130. FIG. 3 additionally illustrates a yoke 170 attached to the hindfoot of a subject undergoing ankle distraction. Weights 195 may be attached or associated with the stand 120 to provide additional stability to the stand 120 and prevent it from tipping toward the subject.

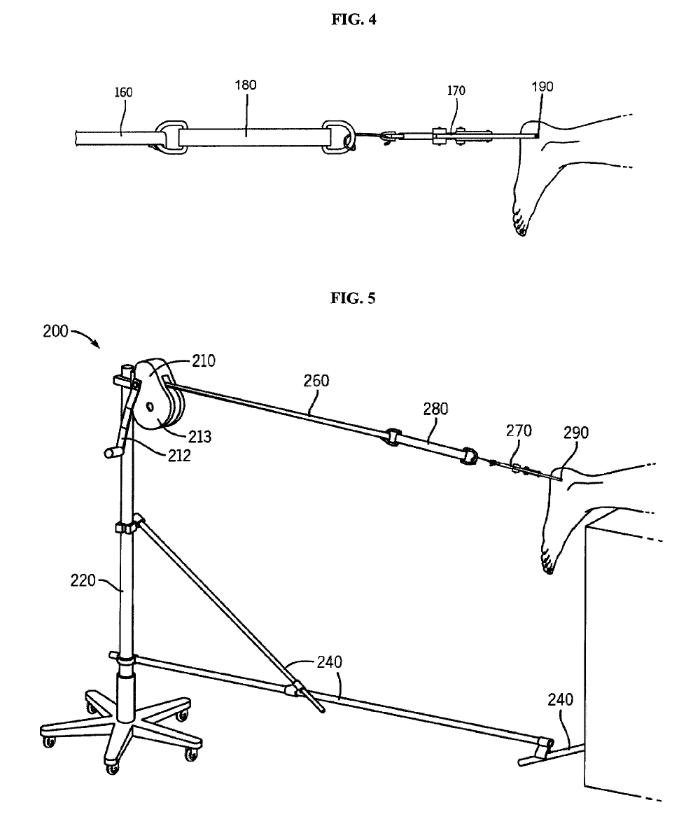

Referring now to FIG. 4, a pin 190 is shown inserted through a bone in the hindfoot of a subject. The pin 190 is attached to both the yoke 170, and the tension line 160, with the tension line 160 transmitting the tension/force to the yoke 170 and the pin 190. A secondary connector 180 is also shown connecting the yoke 170 to the tension line 160.

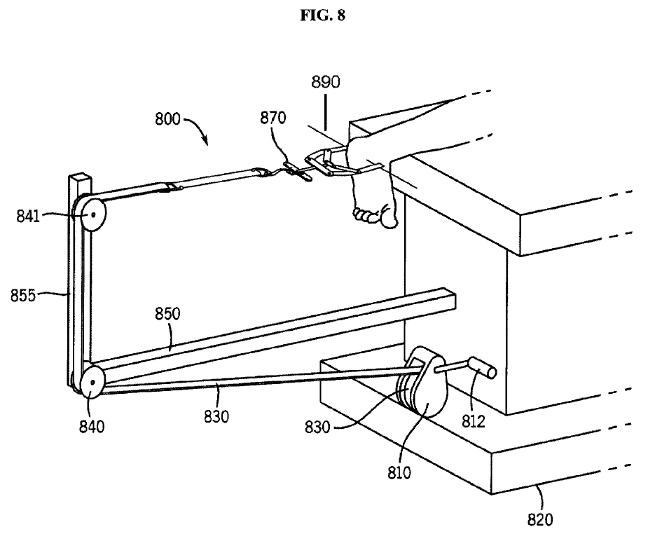

Referring now to FIG. 5, another embodiment of a joint distractor 200 is shown. The exemplified joint distractor 200 has a tensioning device that is a winch 210 having a handle 212 and a spindle 213. The joint distractor 200 also has a stand 220 that is connected to the surgical table via connector beams 240. A tension line 260 connects the winch 210 to the yoke 270 via a secondary connector 280. The yoke 270 attaches to pin 290. In FIG. 8, a different view of a pin 890 is shown.

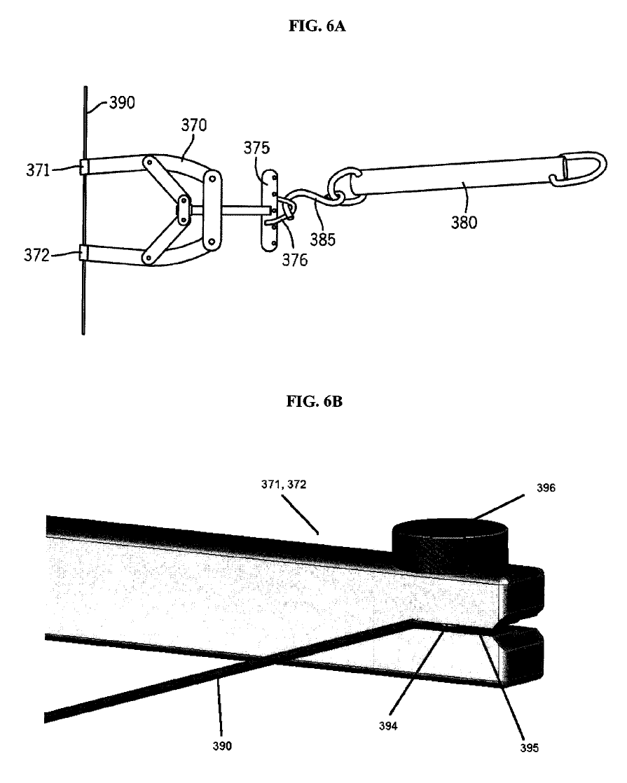

FIG. 6A illustrates one embodiment of a yoke, also known as a traction bow 370. The bow has a generally U-shaped configuration. The bow is connected to wire 390 at a first end 371 of the U-shape and at a second end 372 of the U-shape. A secondary connector 380 is also shown for connection of the traction bow 370 to a tensioning device as described above. The connection of the traction bow 370 to the secondary connector 380 is facilitated by a hook 385, or other suitable connector, and a loop 376 secured to plate 375.

The wire 390 maybe attached to the yoke 370 at the ends 371, 372 via one of many methods known in the art. For example, the ends 371, 372 may be attached to the wire 390 via crimping, via a friction fit of the wire between the head of a bolt and the yoke, or via a friction fit between the wire and a nut and the yoke, via a friction fit with a toothed clamp, although may other such attachments/connections are known. One such other attachments/connections is illustrated in FIG. 6B as a detailed view of one the ends 371, 372 of the traction bow 370. In such embodiments, the wire 390 is inserted into a slot 395. A screw 394 is transversely inserted through the ends such that when the screw 394 is turned the slot 395 is compressed onto the wire 390, thereby securing wire to the traction bow 370. The screw 394 may optionally have a thumb tightening portion 396 as illustrated in FIG. 6B, or the screw 394 may have any type of drive head or other drive mechanism known to those of skill in the art. In some embodiments, the ends of the wire may be shaped to engage the slot with greater surface area and holding strength. In other embodiments, the ends of the wire and the slot are shaped complementary to engage one another to provide greater surface area contact between the wire and the slot as compared to if a round wire were used, thereby increasing the holding strength of the connection.

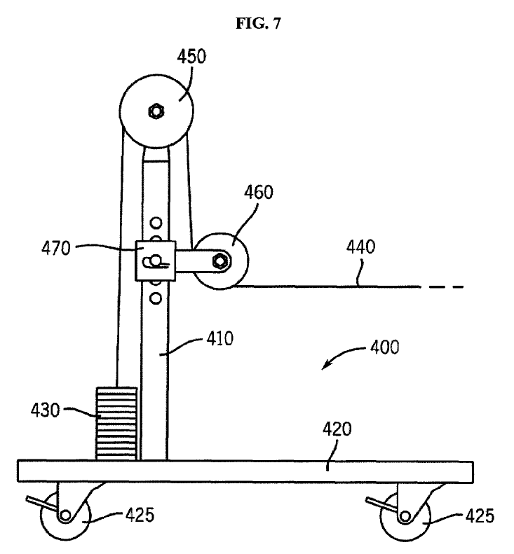

FIG. 7 illustrates one embodiment of a weight and pulley system. The weight and pulley system consists of a stand 400 having an upright member 410, a base 420, weights 430, a tension line 440, and at least one pulley 450. The tension line 440 extends from a point on one a side of the stand 400, such that when the weights 430 are attached to the tension line 440, a force is directed over the at least one pulley 450 and to a pin or wire connected to a bone in the subject, as described above. The relative height position of the at least one pulley 450 may be adjusted vertically. Alternatively, and as shown in FIG. 7, an additional pulley 460 may be in line with the tension line 440 such that the additional pulley 460 is attached to the upright member 410 via a connector 470. The connector 470 may be capable of vertical adjustment of the at least one additional pulley 460. The weight and pulley system may be supported with wheels 425 or legs. If the weight and pulley system is supported by wheels 425, the wheels 425 are lockable wheels to prevent the movement of the apparatus during a surgical procedure.

Referring now to FIG. 8, another embodiment of a joint distractor is shown, where a tensioning device 810 is attached to a surgical table 820. The exemplified joint distractor 800 has a tensioning device 810 and a bar to translate a force from the tensioning device 810 to a patient. The joint distractor 800 is mounted to the surgical table 820. The tensioning of a tension line 830, from the tensioning device 810 to the joint that is to be distracted, is directed by a series of pulleys 840 and 841. The tension line 830 connects the tensioning device 810 to the yoke/traction bow 870, which in turn is connected to the pin 890. In some embodiments, the tensioning device 810 is a winch having a handle 812 and a spindle 813.

The pulleys 840, 841 are connect to a bar having, as exemplified, a horizontal component 850 and a vertical component 855. Optionally, a single angled component may take the place of both horizontal 850 and vertical 855 components. The bar provides support for the pulleys 840, 841 to be fixedly or adjustably positioned. The bar may also have one or more joints included for positioning purposes. For example, one or more joints that will allow the bar to move in one or more vertical or horizontal planes may be incorporated such that precise direction of the force from the tensioning device may be achieved. While FIG. 8 illustrates one attachment of a tensioning device 810 to a surgical table 820, other attachment points are a choice of design and may include various points on, or under, the table, or on the railings or other table or bed structures. Additional stabilizing structures may also be added to the bar to prevent bending or to further stabilize the bar against movement once tension is applied with the tensioning device.

With respect to the pulleys 840, 841, in some embodiments, only a single pulley is necessary, while in other embodiments, more than two pulleys may be used. In other embodiments, the pulleys are not actually pulleys at all, but rather are surfaces on which the tension line 830 may slide to re-direct the force. Placement of the tensioning device 810 on the surgical table 820 may vary depending upon the particular table, or joint that is to be distracted.

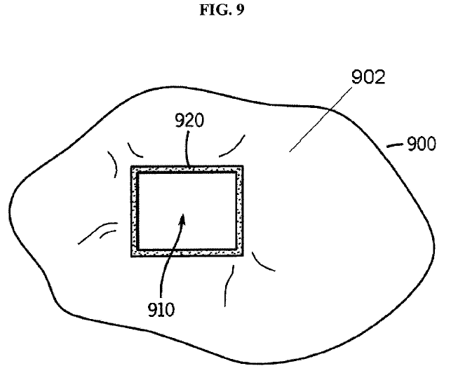

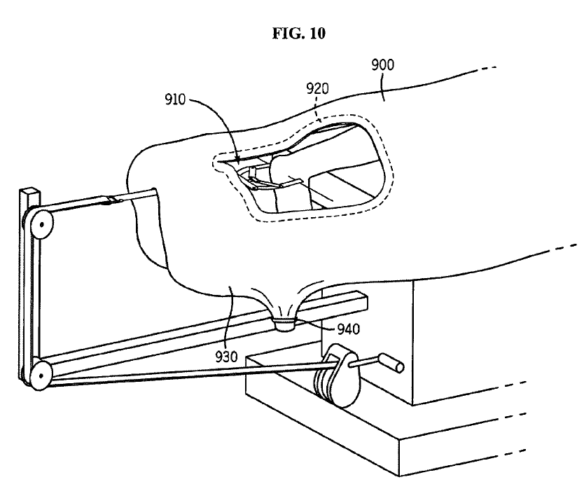

In another aspect, a surgical drape 900 is provided, as illustrated in FIG. 9. The surgical drape 900 has first face 901 (not shown in FIG. 9, but is the opposite side of the drape from the second face), a second face 902, and a cut-out surgical field 910. The surgical field is defined by an adhesive region 920 on the second face 902 in an area proximal to the cut-out surgical field 910. The cut-out surgical field 910 is off-set with respect to the center of the drape. The surgical drape 900 is configured to be placed over and around the area surrounding the surgical site and draping the surgical site from non-sterile areas, as illustrated by FIG. 10. For example, as shown, the lower leg, foot, yoke, and secondary connector are sterile areas, whereas those areas outside of the surgical field may or may not be sterile. The surgical drape may cover the patient as well. In draping the surrounding area, at least a portion of the surgical drape 900 may be gathered under the surgical field by a tie 940 such that at least a portion of the second face 902 forms a basin 930. In the methods described below, the basin 930 is configured to collect fluids from the surgical field so that the surrounding area is not contaminated. The basin 930 is configured to either hold the fluids until completion of the surgical procedure, or a hose or tube may be inserted to remove the fluid, to a collection vessel, as it collects in the basin 930. Alternatively a fluid transfer line such as hose or tube may be attached at the point in which the edges of the surgical drape 900 gather to effectively form a drain in the basin 930. The adhesive region both secures the drape to the patient or surgical apparatuses and helps to maintain the sterility of the surgical field. Adhesives that may be used are known throughout the medical industry.

FIGS. 1-8 and 10 all exemplify ankle distraction, however these general principles may be applied to other joint distractions such as knee, hand, foot, hip, shoulder, elbow, finger, wrist, or other joints. Those of skill will readily envision the exemplified general principles as applied to each of these other joints.

In another aspect, a method of using a medical apparatus embodied herein is provided for the distraction of a joint. In one embodiment, the method includes inserting a pin or a wire through a bone, connecting a tensioning device to the wire or pin with a tension line, and applying a force to the wire or pin until ajoint of a subject is distracted. The bone is typically associated with the joint to be distracted. For example, in the case where the joint to be distracted is an ankle or other subtalar joint, the bone is a bone of the hindfoot such as a talus or a calcaneus.

In another embodiment, once the joint is distracted, the surgeon or medical professional treats or examines the joint. In other cases, the force may then be released to re-engage the joint. In some embodiments, the force is release by unlocking the locking mechanism.

In another embodiment, once the joint is distracted, the surgeon or medical professional treats or examines the joint, and then a cage system, or fixator frame, is attached to the foot, ankle, and/or leg of the subject, thereby keeping the ankle distracted while the bones in the ankle heal. Such cage systems, or fixator frames, are known to those of skill in the art. In such cases, the ankle joint is then allowed to re-engage when the cage system, or fixator frame, is removed at a later date.

In another embodiment, the stand is adjusted by the surgeon or other medical professional to a predetermined distance from the surgical table. The stand may also be adjusted to a predetermined height.

In some embodiments of the methods, the subject is secured to the surgical table to prevent unintended re-engagement of the joint by the subject moving toward to the tensioning device as a result of the applied force.

In another aspect, the surgical drape 900 described above is used in a method of distracting a joint. In such methods the surgical area, or field, is defined by the surgical drape 900 and cut-out surgical field 910. In some embodiments, the method includes draping the surgical drape 900 around the joint to be distracted, gathering the edges of the surgical drape 900, and securing the edges of the surgical drape 900 together with a tie 940 to form a basin 930. The methods may also include inserting a hose or tube into the basin 930 to remove fluids as they are collected. Alternatively, the methods may include attaching a hose or tube at the point in which the edges of the surgical drape 900 gather, thereby forming a drain in the basin 930.

One skilled in the art will readily realize that all ranges and ratios discussed can and do necessarily also describe all subranges and subratios therein for all purposes and that all such subranges and subratios also form part and parcel of this invention. Any listed range or ratio can be easily recognized as sufficiently describing and enabling the same range or ratio being broken down into at least equal halves, thirds, quarters, fifths, tenths, etc. As a non-limiting example, each range or ratio discussed herein can be readily broken down into a lower third, middle third and upper third, etc.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

While several, non-limiting examples have been illustrated and described, it should be understood that changes and modifications can be made therein in accordance with ordinary skill in the art without departing from the invention in its broader aspects as defined in the following claims.

What is claimed is:

1. A medical apparatus comprising:
a tensioning device;
a wire or pin configured to be inserted transversely through a bone; and
a tension line configured to connect the tensioning device to the wire or pin;
wherein, the tension line transmits a force from the tensioning device to the wire or pin to facilitate distraction of a joint of a subject.

2. The medical apparatus of claim 1, wherein the tensioning device is attached to a fixed point.

3. The medical apparatus of claim 2, wherein the fixed point is a surgical table upon which the subject is positioned; an attachment to the surgical table upon which the subject is positioned; a stand; a floor; a ceiling; or a wall.

4. The medical apparatus of claim 2, wherein the fixed point is a stand and the stand is connected to a surgical table upon which the subject is positioned; a floor; a ceiling; or a wall.

5. The medical apparatus of claim 1, wherein the tensioning device comprises a pulley, a manual winch, an electronically driven winch, a pneumatic piston, a pneumatic winch, a hydraulic piston, a hydraulic winch, a block-and-tackle, a weight system, or a combination of any two or more thereof.

6. The medical apparatus of claim 1, wherein the tensioning device comprises a locking mechanism.

7. The medical apparatus of claim 6, wherein the locking mechanism is a ratchet or a clamp.

8. The medical apparatus of claim 1, wherein the joint is a joint of the ankle or foot.

9. The medical apparatus of claim 1, wherein the joint is a hip.

10. The medical apparatus of claim 1, wherein the joint is a joint of the knee, shoulder, elbow, foot, toe, hand, finger, or wrist.

11. The medical apparatus of claim 8, wherein the bone is a bone of the ankle or foot.

12. The medical apparatus of claim 11, wherein the bone foot is a talus or a calcaneus.

13. The medical apparatus of claim 9, wherein the bone is a bone of the leg.

14. The medical apparatus of claim 13, wherein the bone of the leg is a femur.

15. The medical apparatus of claim 1, further comprising a clamp connecting the tension line to the wire or pin.

16. The medical apparatus of claim 15, wherein the clamp is a yoke, a traction bale, or a traction bow.

17. A method comprising distracting a joint using the medical apparatus of claim 1, comprising:
inserting the wire or pin transversely through the bone;
connecting the wire or pin to the tension line; and
tensioning the tension line to distract the joint of the subject.

18. The method of claim 17 further comprising securing the tensioning device to the fixed point.

19. The method of claim 17, wherein the tensioning device comprises a pulley, a manual winch, an electronic winch, a block-and-tackle, a weight system, or a combination of any two or more thereof.

20. The method of claim 17 further comprising securing the subject to a surgical table.

21. The method of claim 17 further comprising connecting the wire or pin to a clamp and connecting the clamp to the tension line.

22. A surgical drape comprising a cut-out surgical field, a first face and a second face, the second face comprising an adhesive region proximal to the cut-out surgical field.

23. The surgical drape of claim 22, wherein the drape is configured to be gathered and tied under a surgical site such that the second face forms a basin.

24. The surgical drape of claim 22, further comprising a fluid transfer line.
